(19) United States
(12) Patent Application Publication
(10) Pub. No.: US 2025/0143619 A1
(43) Pub. Date: May 8, 2025

(54) PREDICTION SYSTEM OF SIGNIFICANT CONGENITAL HEART DISEASE IN INFANTS AND OPERATION METHOD THEREOF AND NON-TRANSITORY COMPUTER READABLE MEDIUM

(71) Applicants: Chang Gung Memorial Hospital, Linkou, Taoyuan City (TW); Taipei Medical University (TMU), Taipei City (TW)

(72) Inventors: Syu-Jyun Peng, Zhubei City (TW); Yu-Shin Lee, Taoyuan City (TW)

(73) Assignees: Chang Gung Memorial Hospital, Linkou, Taoyuan City (TW); Taipei Medical University (TMU), Taipei City (TW)

(21) Appl. No.: 18/643,235

(22) Filed: Apr. 23, 2024

(30) Foreign Application Priority Data
Nov. 3, 2023 (TW) 112142533

(51) Int. Cl. A61B 5/318; A61B 5/00

(52) U.S. Cl. CPC A61B 5/318; A61B 5/726; A61B 5/7267; A61B 2503/045

(57) ABSTRACT

The present disclosure provides an operation method of a prediction system of significant congenital heart disease in infants in infants, which includes steps as follows. The continuous wavelet transformation is performed on the electrocardiogram to obtain the processed electrocardiogram; the processed electrocardiogram is oversampled to obtain multiple electrocardiogram segments; the transfer learning through multiple pre-trained models based on the multiple electrocardiogram segments is used to establish a significant congenital heart disease model.

### RELATED APPLICATION

This application claims priority to Taiwan Patent Application No. 112142533, filed Nov. 3, 2023, the entirety of which is herein incorporated by reference.

### BACKGROUND

### Field of Invention

The present invention relates to systems and operation methods, and more particularly, prediction systems of significant congenital heart disease in infants and operation methods thereof.

### Description of Related Art

The severity of congenital heart disease is the main consideration in determining treatment methods. Congenital heart disease could be further classified into left heart and right heart disease according to its hemodynamic influence on heart chambers. Only significant congenital heart disease requires early interventional treatment.

The current standard diagnosis of congenital heart disease is cardiac ultrasound, which requires experienced operators and expensive machines. Currently, the cardiac ultrasound can only be performed in medical centers and large regional hospitals. In addition, cardiac ultrasound screening performed at birth often detects tiny lesions of patent foramen ovale and peripheral pulmonary stenosis; however, most of these lesions recover spontaneously.

Currently, the most commonly used screening method is blood oxygen concentration testing after birth. Although the cost is low and the operation is convenient, the blood oxygen concentration testing is easily affected by the condition of the lung lesions of the newborn before and after birth. The blood oxygen concentration testing can only detect congenital heart disease that can cause cyanosis. The blood oxygen concentration testing cannot detect congenital heart disease that does not cause cyanosis but is enough to cause cardiac failure of significant congenital heart disease. The detection of diseases that do not require treatment, such as the patent foramen ovale and the peripheral pulmonary stenosis, does not help the patient, but instead affects the patient's life, such as insurance applications.

The electrocardiogram changes in children with congenital heart disease are mainly caused by the hemodynamic influence caused by the lesions, which are observed from the signals of heart axis changes and ventricular dilation. However, in infants and young children with congenital heart disease, the electrocardiogram is more likely to fall within the seemingly normal range due to the shorter cardiac remodeling time.

### SUMMARY

In one or more various aspects, the present disclosure is directed to identifying and quantizing systems and operation methods thereof.

An embodiment of the present disclosure is related to a prediction system of a significant congenital heart disease in infants. The prediction system includes a storage device and a processor. The storage device is configured to store at least one instruction and at least one electrocardiogram, and an original format of the at least one electrocardiogram is a first format. The processor is coupled to the storage device, and the processor configured to access and execute the at least one instruction for: converting a first format into a second format, so that the at least one electrocardiogram has a second format; performing a continuous wavelet transformation on the at least one electrocardiogram having the second format to obtain at least one processed electrocardiogram; performing an oversampling on the at least one processed electrocardiogram to obtain a plurality of electrocardiogram segments; using a transfer learning through a plurality of pre-trained models based on the plurality of the electrocardiogram segments to establish a significant congenital heart disease model.

In one embodiment of the present disclosure, the first format is an extensible markup language format, and the second format is a comma-separated values format.

In one embodiment of the present disclosure, the oversampling divides the at least one processed electrocardiogram into the plurality of electrocardiogram segments in each time unit of a predetermined period.

In one embodiment of the present disclosure, the number of electrocardiogram segments is five times the number of processed electrocardiograms, and the processor accesses and executes the at least one instruction for: performing a five-fold cross validation on the plurality of the electrocardiogram segments, thereby ensuring a stability of the plurality of the pre-trained models.

In one embodiment of the present disclosure, the processor accesses and executes the at least one instruction for: training the plurality of the pre-trained models through the transfer learning to obtain a plurality of trained models; and selecting one trained model with a highest accuracy rate from the plurality of trained models, so as to designate the one trained model as the significant congenital heart disease model.

Another embodiment of the present disclosure is related to an operation method of a prediction system of a significant congenital heart disease in infants. The operation method includes steps of: performing a continuous wavelet transformation on at least one electrocardiogram to obtain at least one processed electrocardiogram; performing an oversampling on the at least one processed electrocardiogram to obtain a plurality of electrocardiogram segments; and using a transfer learning through a plurality of pre-trained models based on the plurality of the electrocardiogram segments to establish a significant congenital heart disease model.

In one embodiment of the present disclosure, an original format of the at least one electrocardiogram is a first format, and the step of performing the continuous wavelet transformation on the at least one electrocardiogram to obtain the at least one processed electrocardiogram includes: converting the first format into a second format, so that the at least one electrocardiogram has a second format; and performing the continuous wavelet transformation on the at least one electrocardiogram having the second format to obtain at least one processed electrocardiogram.

In one embodiment of the present disclosure, the step of performing the oversampling on the at least one processed electrocardiogram to obtain the plurality of electrocardiogram segments includes: dividing the at least one processed electrocardiogram into the plurality of electrocardiogram segments in each time unit of a predetermined period.

In one embodiment of the present disclosure, the step of using the transfer learning through the plurality of pre-trained models based on the plurality of the electrocardiogram segments to establish the significant congenital heart disease model includes: performing a five-fold cross validation on the plurality of the electrocardiogram segments, thereby ensuring a stability of the plurality of the pre-trained models, wherein a number of electrocardiogram segments is five times a number of processed electrocardiograms.

In one embodiment of the present disclosure, the step of using the transfer learning through the plurality of pre-trained models based on the plurality of the electrocardiogram segments to establish the significant congenital heart disease model includes: training the plurality of the pre-trained models through the transfer learning to obtain a plurality of trained models; and selecting one trained model with a highest accuracy rate from the plurality of trained models, so as to designate the one trained model as the significant congenital heart disease model.

Technical advantages are generally achieved, by embodiments of the present disclosure. With the prediction system of the significant congenital heart disease in infants and its operation method of the present disclosure, the significant congenital heart disease model can effectively identify different degrees of congenital heart disease. By inputting electrocardiogram data into the significant congenital heart disease model, the significant congenital heart disease model can predict whether infants aged 0 to 5 years have significant congenital heart disease.

Many of the attendant features will be more readily appreciated, as the same becomes better understood by reference to the following detailed description considered in connection with the accompanying drawings.

### BRIEF DESCRIPTION OF THE DRAWINGS

The invention can be more fully understood by reading the following detailed description of the embodiment, with reference made to the accompanying drawings as follows:

FIG. 1 is a block diagram of a prediction system of significant congenital heart disease in infants in infants according to one embodiment of the present disclosure;

FIG. 2 is a flow chart of a classification of congenital heart disease according to one embodiment of the present disclosure; and

FIG. 3 is a flow chart of an operation method of a prediction system of significant congenital heart disease in infants according to one embodiment of the present disclosure.

### DETAILED DESCRIPTION

Reference will now be made in detail to the present embodiments of the invention, examples of which are illustrated in the accompanying drawings. Wherever possible, the same reference numbers are used in the drawings and the description to refer to the same or like parts.

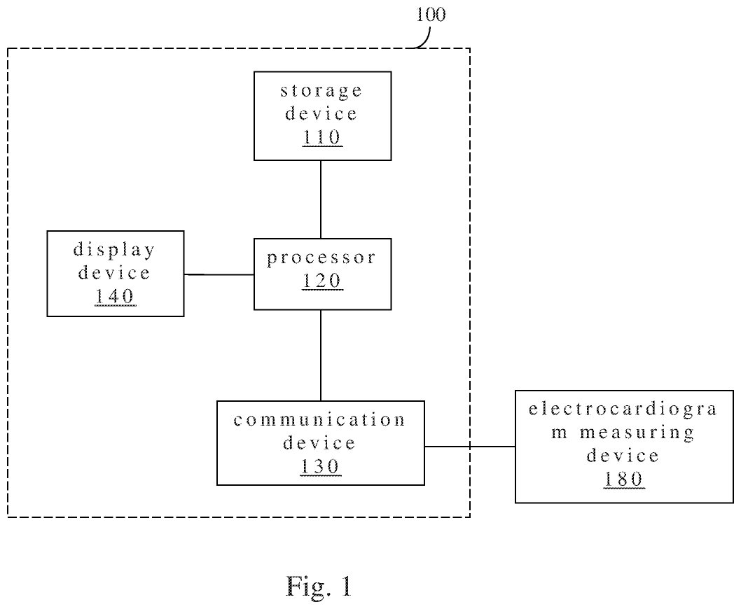

Referring to FIG. 1, in one aspect, the present disclosure is directed to a prediction system 100 of significant congenital heart disease in infants in infants. The prediction system 100 of significant congenital heart disease in infants may be easily integrated into a computer and may be applicable or readily adaptable to all technologies. Technical advantages are generally achieved by the prediction system 100 of significant congenital heart disease in infants according to embodiments of the present disclosure. Herewith the prediction system 100 of significant congenital heart disease in infants is described below with FIG. 1.

The subject disclosure provides the prediction system 100 of significant congenital heart disease in infants in accordance with the subject technology. Various aspects of the present technology are described with reference to the drawings. In the following description, for purposes of explanation, numerous specific details are set forth in order to provide a thorough understanding of one or more aspects. It can be evident, however, that the present technology can be practiced without these specific details. In other instances, well-known structures and devices are shown in block diagram form in order to facilitate describing these aspects. The word “exemplary” is used herein to mean “serving as an example, instance, or illustration.” Any embodiment described herein as “exemplary” is not necessarily to be construed as preferred or advantageous over other embodiments.

In practice, for example, the prediction system 100 of significant congenital heart disease in infants can be a computer server. The computer server can be remotely managed in a manner that substantially provides accessibility, consistency, and efficiency. Remote management removes the need for input/output interfaces in the servers. An administrator can manage a large data centers containing numerous rack servers using a variety of remote management tools, such as simple terminal connections, remote desktop applications, and software tools used to configure, monitor, and troubleshoot server hardware and software.

As used herein, “around”, “about”, “substantially” or “approximately” shall generally mean within 20 percent, preferably within 10 percent, and more preferably within 5 percent of a given value or range. Numerical quantities given herein are approximate, meaning that the term “around”, “about”, “substantially” or “approximately” can be inferred if not expressly stated.

In practice, in an embodiment of the present disclosure, the prediction system 100 of significant congenital heart disease in infants can selectively establish a connection with the electrocardiogram measuring device 180. It should be understood that in the embodiments and the scope of the patent application, the description involving “connection” can generally refer to a component that indirectly communicates with another component by wired and/or wireless communication through another component, or a component that is physically connected to another element without through another element. For example, the prediction system 100 can indirectly communicate with the electrocardiogram measuring device 180 through wired and/or wireless communication via another component, or the prediction system 100 can be physically connected to the electrocardiogram measuring device 180 without another component. Those with ordinary skill in the art may select the connection manner depending on the desired application.

FIG. 1 is a block diagram of the prediction system 100 of significant congenital heart disease in infants according to one embodiment of the present disclosure. As shown in FIG. 1, the prediction system 100 of significant congenital heart disease in infants includes a storage device 110, a processor 120, a communication device 130 and a display device 140. For example, the storage device 110 can be a hard drive, a flash memory or another storage device, the processor 120 can be a central processing unit, the communication device 130 can be a wired and/or wireless network device, and the display device 140 can be a built-in display or an external screen.

In structure, the storage device 110 is electrically connected to the processor 120, the processor 120 is electrically connected to the communication device 130 and the display device 140. It should be noted that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. For example, the storage device 110 may be a built-in storage device that is directly connected to the processor 120, or the storage device 110 may be an external storage device that is indirectly connected to the processor 120 through the network device.

In some embodiments of the present disclosure, the storage device 110 is configured to store at least one instruction and at least one electrocardiogram, and an original format of the at least one electrocardiogram is a first format. The processor 120 is configured to access and execute the at least one instruction for: converting a first format into a second format, so that the at least one electrocardiogram has a second format; performing a continuous wavelet transformation on the at least one electrocardiogram having the second format to obtain at least one processed electrocardiogram; performing an oversampling on the at least one processed electrocardiogram to obtain a plurality of electrocardiogram segments; using a transfer learning through a plurality of pre-trained models based on the plurality of the electrocardiogram segments to establish a significant congenital heart disease model. The significant congenital heart disease model can effectively identify different degrees of congenital heart disease. By inputting electrocardiogram data into the significant congenital heart disease model, the significant congenital heart disease model can predict whether infants aged 0 to 5 years have significant congenital heart disease.

Regarding the above-mentioned first format and second format, in some embodiments of the present disclosure, the first format is an extensible markup language (XML) format, and the second format is a comma-separated values (CSV) format. In practice, the electrocardiogram with the second format is more conducive to the continuous wavelet transformation. For example, the subject's 10-second 12-lead electrocardiogram data was exported to an extensible markup language format electrocardiogram with a sampling rate of 500 Hz by the electrocardiogram measuring device 180. The processor 120 creates a python programming language environment to converts the extensible markup language format electrocardiogram into a comma-separated values format electrocardiogram, and the processor 120 executes the MATLAB software to using the Morlet waveform as the mother wave to perform the continuous wavelet transformation in every time unit of 2 seconds for converting the comma-separated values format electrocardiogram into the 12×1000 matrix stored in the mat format for analysis.

Regarding the above oversampling, in some embodiments of the present disclosure, the oversampling can divide the at least one processed electrocardiogram into the plurality of electrocardiogram segments in each time unit of a predetermined period. In practice, for example, the overall duration of processed electrocardiogram is about 10 seconds, and the predetermined period is about 2 seconds; after segmentation, each patient has 5 samples (i.e., electrocardiogram segments), which indicate five times the sample increment (i.e., 5-times oversampling).

Following the above, in some embodiments of the present disclosure, the number of electrocardiogram segments is five times the number of processed electrocardiograms, and the processor 120 accesses and executes the at least one instruction for: performing a five-fold cross validation on the plurality of the electrocardiogram segments, thereby ensuring the stability of the plurality of the pre-trained models, so as to be conducive to the transfer learning to train the pre-trained models.

Regarding the above-mentioned pre-trained models, in some embodiments of the present disclosure, the processor 120 accesses and executes the at least one instruction for: training the plurality of the pre-trained models through the transfer learning to obtain a plurality of trained models; and selecting one trained model with a highest accuracy rate from the plurality of trained models, so as to designate the one trained model as the significant congenital heart disease model. By performing the transfer learning on different pre-trained models, the finally selected significant congenital heart disease model has the better discrimination ability.

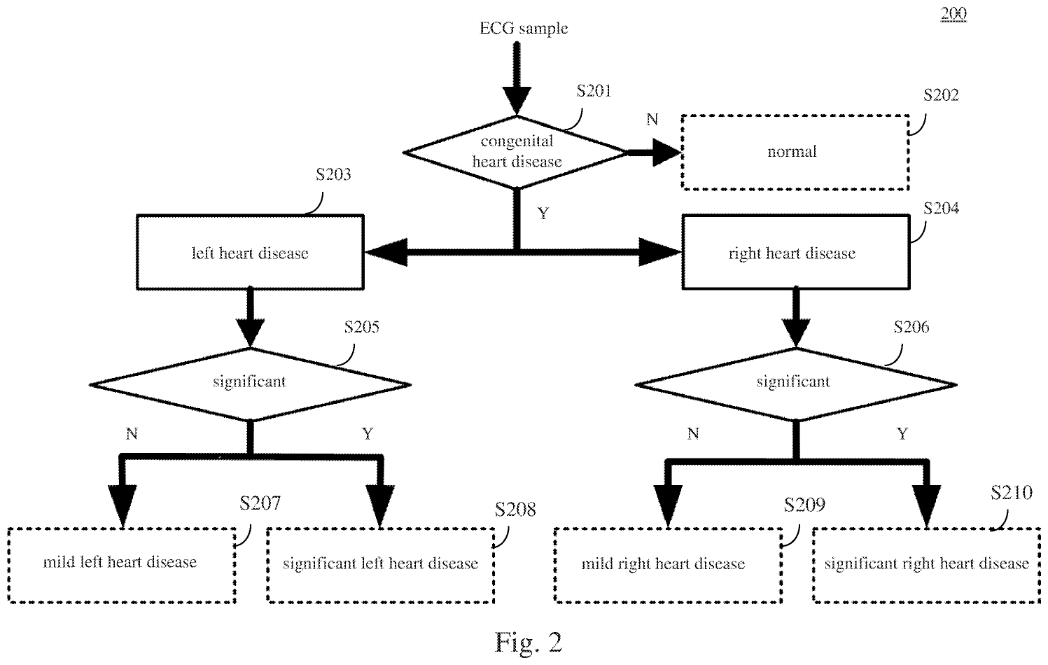

For a more complete understanding of the classification of congenital heart disease, referring FIGS. 1-2, FIG. 2 is a flow chart of the classification of congenital heart disease according to one embodiment of the present disclosure. It should be noted that the electrocardiogram (ECG) sample in FIG. 2 can be medical record data with or without congenital heart disease, which is stored in the storage device 110. The processor 120 can quickly performs the classification on the congenital heart disease status corresponding to the electrocardiogram with the above-mentioned first format according to the medical record data with congenital heart disease, so as to facilitate the training and verification of transfer learning.

In step S201, it is determined whether the electrocardiogram is classified as congenital heart disease. When the electrocardiogram is classified as no heart disease, in step S202, the electrocardiogram is classified as normal (e.g., no heart disease).

When the electrocardiogram is classified as congenital heart disease, in step S203, the congenital heart disease is left heart disease. In step S205, it is determined whether the left heart disease is significant. When the left heart disease is not significant, in step S207, the left heart disease is determined to be a mild left heart disease (e.g., small ventricular septal defect, small patent ductus arteriosus, mild aortic valve stenosis and mild coarctation of aorta, etc.). When the left heart disease is significant, in step S208, the left heart disease is determined to be a significant left heart disease (e.g., large ventricular septal defect, large patent ductus arteriosus, severe aortic valve stenosis, severe coarctation of aorta, etc.).

When the electrocardiogram is classified as congenital heart disease, in step S204, the congenital heart disease is right heart disease. In step S206, it is determined whether the right heart disease is significant. When the right heart disease is not significant, in step S209, the right heart disease is determined to be a mild right heart disease (e.g., mild atrial defect, mild pulmonary valve stenosis, etc.). When the right heart disease is significant, in step S210, the right heart disease is determined to be a significant right heart disease (e.g., large atrial septal defect, severe pulmonary valve stenosis, Tetralogy of Fallot, etc.).

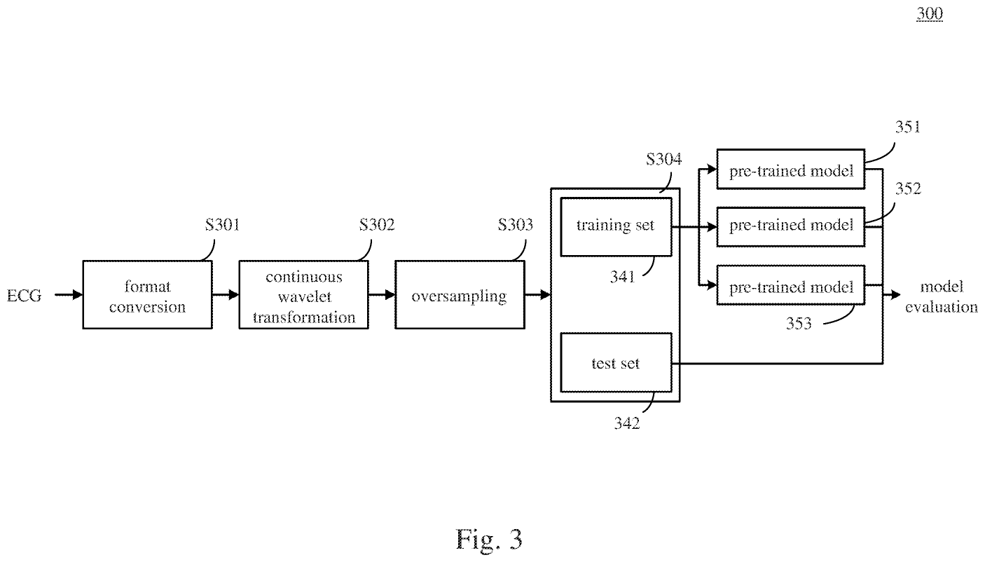

For a more complete understanding of an operation method of the a prediction system 100 of significant congenital heart disease in infants, referring FIGS. 1-2, FIG. 3 is a flow chart of the operation method 300 of the prediction system 100 of significant congenital heart disease in infants according to one embodiment of the present disclosure. As shown in FIG. 3, the operation method 300 includes operations S301-S304. However, as could be appreciated by persons having ordinary skill in the art, for the steps described in the present embodiment, the sequence in which these steps are performed, unless explicitly stated otherwise, can be altered depending on actual needs; in certain cases, all or some of these steps can be performed concurrently.

The operation method 300 may take the form of a computer program product on a computer-readable storage medium having computer-readable instructions embodied in the medium. Any suitable storage medium may be used including non-volatile memory such as read only memory (ROM), programmable read only memory (PROM), erasable programmable read only memory (EPROM), and electrically erasable programmable read only memory (EEPROM) devices; volatile memory such as SRAM, DRAM, and DDR-RAM; optical storage devices such as CD-ROMs and DVD-ROMs; and magnetic storage devices such as hard disk drives and floppy disk drives.

In steps S301 and S302, the continuous wavelet transformation is performed on the electrocardiogram to obtain a processed electrocardiogram. Specifically, in some embodiments of the present disclosure, the original format of the electrocardiogram is the first format (e.g., an extensible markup language format). In step S301, the first format is converted into the second format, so that the electrocardiogram has the second format. Compared with other formats, the second format (e.g., a comma-separated values format) is conducive to the continuous wavelet transformation.

In step S302, the continuous wavelet transformation is performed on the electrocardiogram with the second format to obtain a processed electrocardiogram. The continuous wavelet transformation is a time-frequency analysis tool that uses inner products to measure the correlation between signals and analysis functions. In practice, compared with general wavelet transformation, the continuous wavelet transformation is more conducive to the processes of the electrocardiogram waveforms whose frequency changes over time.

In step S303, the oversampling is performed on the processed electrocardiogram obtain a plurality of electrocardiogram segments. Specifically, in some embodiments of the present disclosure, in step S303, the processed electrocardiogram is divided into a plurality of electrocardiogram segments in each time unit of a predetermined period. In practice, for example, the overall duration of processed electrocardiogram is about 10 seconds, and the predetermined period is about 2 seconds; after segmentation, each patient has 5 samples (i.e., electrocardiogram segments), which indicate five times the sample increment (i.e., 5-times oversampling).

Next, in step S304, using a transfer learning through a plurality of pre-trained models 251 to 253 based on the plurality of the electrocardiogram segments to establish a significant congenital heart disease model. Specifically, since the overall duration of the processed electrocardiogram is five times the predetermined time, the number of the electrocardiogram segments is five times the number of processed electrocardiograms. In some embodiments of the present disclosure, in step S304, a five-fold cross validation is performed on the electrocardiogram segments, thereby ensuring the stability of the pre-trained models 251 to 253.

In the training set 341, when there is an obvious imbalance in the data (i.e., electrocardiograms) between each category (e.g., normal, mild left heart disease, significant left heart disease, mild right heart disease and significant right heart disease), the data increment is used to reduce the data imbalance. The test set 342 does not uses the data increment to ensure the accuracy of verification. The classification of the five-fold cross-validation (e.g., the classification in FIG. 2) is executed by processor 120 according to the sample serial number corresponding to the electrocardiogram to ensure that electrocardiogram segments from the same sample cannot be divided into the training set 341 and the test set 342 at the same time. For example, processor 120 can set multiple sets of consecutive sample serial numbers to be classified into multiple different categories.

The pre-trained models 251 to 253 are trained through the transfer learning to obtain a plurality of trained models; one trained model with a highest accuracy rate is selected from the plurality of trained models, so as to designate the one trained model as the significant congenital heart disease model.

In practice, for example, the pre-trained models 251 can be Resnet-18, which is a convolutional neural network with 18 layers, trained from millions of images in the ImageNet database. The feature of Resnet-18 is the use of residual learning to solve the degradation problem in deep learning.

The pre-trained models 252 can be Inceptionresnet-V2, which is developed by Google and is a convolutional neural network with 164 layers. The network of Inceptionresnet-V2 can converge faster and be more stable during training.

The pre-trained models 253 can be Nasnetmobile, which was proposed by scientists working at Google Brain in 2018. Its characteristic is that the architectural design part is also obtained by computing the neural network search architecture.

In practice, for example, after the above-mentioned Resnet-18, Inceptionresnet-V2 and Nasnetmobile are trained, the highest accuracy rate model is the trained Resnet-18 that serves as the significant congenital heart disease model.

In a controlled experiment, the time series Long Short-Term Memory (LSTM) model is used to predict significant congenital heart disease for infants and young children aged 0-5 years old by using twelve-lead electrocardiogram, but the performance of the LSTM model is not good. In addition, the performance of directly using neural networks for classifying electrocardiograms is also poor.

In view of the above, technical advantages are generally achieved, by embodiments of the present disclosure. With the prediction system 100 of the significant congenital heart disease in infants and its operation method 300 of the present disclosure, the significant congenital heart disease model can effectively identify different degrees of congenital heart disease (e.g., normal, mild left heart disease, significant left heart disease, mild right heart disease and significant right heart disease). By inputting electrocardiogram data into the significant congenital heart disease model, the significant congenital heart disease model can predict whether infants aged 0 to 5 years have significant congenital heart disease (e.g., significant left heart disease and/or significant right heart disease).

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A prediction system of a significant congenital heart disease in infants, comprising:
a storage device configured to store at least one instruction and at least one electrocardiogram, and an original format of the at least one electrocardiogram being a first format; and
a processor coupled to the storage device, and the processor configured to access and execute the at least one instruction for:
converting the first format into a second format, so that the at least one electrocardiogram has the second format;
performing a continuous wavelet transformation on the at least one electrocardiogram having the second format to obtain at least one processed electrocardiogram;
performing an oversampling on the at least one processed electrocardiogram to obtain a plurality of electrocardiogram segments; and
using a transfer learning through a plurality of pre-trained models based on the plurality of the electrocardiogram segments to establish a significant congenital heart disease model.

2. The prediction system of the significant congenital heart disease in the infants of claim 1, wherein the first format is an extensible markup language format, and the second format is a comma-separated values format.

3. The prediction system of the significant congenital heart disease in the infants of claim 1, wherein the oversampling divides the at least one processed electrocardiogram into the plurality of electrocardiogram segments in each time unit of a predetermined period.

4. The prediction system of the significant congenital heart disease in the infants of claim 3, wherein a number of electrocardiogram segments is five times a number of processed electrocardiograms, and the processor accesses and executes the at least one instruction for:
performing a five-fold cross validation on the plurality of the electrocardiogram segments, thereby ensuring a stability of the plurality of the pre-trained models.

5. The prediction system of the significant congenital heart disease in the infants of claim 1, wherein the processor accesses and executes the at least one instruction for:
training the plurality of the pre-trained models through the transfer learning to obtain a plurality of trained models; and
selecting one trained model with a highest accuracy rate from the plurality of trained models, so as to designate the one trained model as the significant congenital heart disease model.

6. An operation method of a prediction system of a significant congenital heart disease in infants, and the operation method, comprising steps of:
performing a continuous wavelet transformation on at least one electrocardiogram to obtain at least one processed electrocardiogram;
performing an oversampling on the at least one processed electrocardiogram to obtain a plurality of electrocardiogram segments; and
using a transfer learning through a plurality of pre-trained models based on the plurality of the electrocardiogram segments to establish a significant congenital heart disease model.

7. The operation method of claim 6, wherein an original format of the at least one electrocardiogram is a first format, and the step of performing the continuous wavelet transformation on the at least one electrocardiogram to obtain the at least one processed electrocardiogram comprises:
converting the first format into a second format, so that the at least one electrocardiogram has the second format; and
performing the continuous wavelet transformation on the at least one electrocardiogram having the second format to obtain the at least one processed electrocardiogram.

8. The operation method of claim 6, wherein the step of performing the oversampling on the at least one processed electrocardiogram to obtain the plurality of electrocardiogram segments comprises:
dividing the at least one processed electrocardiogram into the plurality of electrocardiogram segments in each time unit of a predetermined period.

9. The operation method of claim 8, wherein the step of using the transfer learning through the plurality of pre-trained models based on the plurality of the electrocardiogram segments to establish the significant congenital heart disease model comprises:
performing a five-fold cross validation on the plurality of the electrocardiogram segments, thereby ensuring a stability of the plurality of the pre-trained models, wherein a number of electrocardiogram segments is five times a number of processed electrocardiograms.

10. The operation method of claim 6, wherein the step of using the transfer learning through the plurality of pre-trained models based on the plurality of the electrocardiogram segments to establish the significant congenital heart disease model comprises:
training the plurality of the pre-trained models through the transfer learning to obtain a plurality of trained models; and
selecting one trained model with a highest accuracy rate from the plurality of trained models, so as to designate the one trained model as the significant congenital heart disease model.

11. A non-transitory computer readable medium to store a plurality of instructions for commanding a computer to execute an operation method, and the operation method comprising steps of:
performing a continuous wavelet transformation on at least one electrocardiogram to obtain at least one processed electrocardiogram;
performing an oversampling on the at least one processed electrocardiogram to obtain a plurality of electrocardiogram segments; and
using a transfer learning through a plurality of pre-trained models based on the plurality of the electrocardiogram segments to establish a significant congenital heart disease model.

12. The non-transitory computer readable medium of claim 11, wherein an original format of the at least one electrocardiogram is a first format, and the step of performing the continuous wavelet transformation on the at least one electrocardiogram to obtain the at least one processed electrocardiogram comprises:
converting the first format into a second format, so that the at least one electrocardiogram has the second format; and
performing the continuous wavelet transformation on the at least one electrocardiogram having the second format to obtain the at least one processed electrocardiogram.

13. The non-transitory computer readable medium of claim 11, wherein the step of performing the oversampling on the at least one processed electrocardiogram to obtain the plurality of electrocardiogram segments comprises:
dividing the at least one processed electrocardiogram into the plurality of electrocardiogram segments in each time unit of a predetermined period.

14. The non-transitory computer readable medium of claim 13, wherein the step of using the transfer learning through the plurality of pre-trained models based on the plurality of the electrocardiogram segments to establish the significant congenital heart disease model comprises:
performing a five-fold cross validation on the plurality of the electrocardiogram segments, thereby ensuring a stability of the plurality of the pre-trained models, wherein a number of electrocardiogram segments is five times a number of processed electrocardiograms.

15. The non-transitory computer readable medium of claim 11, wherein the step of using the transfer learning through the plurality of pre-trained models based on the plurality of the electrocardiogram segments to establish the significant congenital heart disease model comprises:
training the plurality of the pre-trained models through the transfer learning to obtain a plurality of trained models; and
selecting one trained model with a highest accuracy rate from the plurality of trained models, so as to designate the one trained model as the significant congenital heart disease model.
